(19) United States
(12) Patent Application Publication
(10) Pub. No.: US 2014/0257439 A1
(43) Pub. Date: Sep. 11, 2014

(54) ADJUSTABLE THERAPEUTIC LIGHTS

(71) Applicant: Verilux, Inc., (US)

(72) Inventor: Ryan J. Douglas, Stillwater, MN (US)

(73) Assignee: VERILUX, INC., Waitsfield, VT (US)

(21) Appl. No.: 13/787,246

(22) Filed: Mar. 6, 2013

(51) Int. Cl. F21V 9/10; A61N 5/06

(52) U.S. Cl. CPC F21V 9/10; A61N 5/0618
USPC 607/90; 29/428

(57) ABSTRACT

A light therapy process wherein a patient chooses a filter to adjust a comfort level of the light used for the therapy.

### TECHNICAL FIELD

The Technical Field relates to materials and methods for creating adjustability in lights used for bright light therapy, including seasonal affective disorder (SAD).

### BACKGROUND

Bright light therapy is effective to treat SAD, circadian rhythm disturbances and other conditions. There is a general consensus that treatments for seasonal affective disorder should use a light intensity of about 10,000 lux.

### SUMMARY

The Applicants have been actively involved in light therapy treatment and device design. They have observed that not all eyes respond the same to light provided for light therapy. Patient compliance is a well-known problem in the light therapy arts, as with patients that discontinue use when they feel better or stopping use because of discomfort or inconvenience. Previous approaches have involved changing designs to accommodate various intended placements of the light therapy device, its size, changing its intensity, or making the intensity adjustable to a limited range of preset values. The Applicants, however, have discovered that adjusting the color temperature to best suit each user improves the effectiveness of the therapies, improves compliance, and increases comfort in use.

There is a further discovery that choosing a suitable color temperature allows the user to receive a higher retinal dose of light than would otherwise be possible. Therefore the intensity and size of the lights can be reduced to improve comfort and compliance without sacrificing dosage. The term dose refers to the product of time and intensity. The term intensity refers to a power of the light, and may be measured in lux; the term refers to photopic intensity. The term retinal dose refers to the dose received at the retina. As is explained in more detail, adjusting a color temperature can affect the amount of light that is passed through the pupil to the retina. Certain technical discoveries had to be made so as to implement the solutions described in detail below.

An embodiment of the invention is a light system comprising a lamp that comprises a housing and a light source, and a filter set that comprises at least a first filter and a second filter. The term lamp is broad and refers to luminaires and lights in general. The light source is in the housing and the filters in the filter set are mountable on the housing to filter light emitted from the light source. The housing preferably lets light escape only after passing through a filter. Light emitted from the lamp, after passing through one of the first or second filters, is white light, and has a first color temperature after passing through the first filter when the first filter is mounted on the housing and the light has a second color temperature after passing through the second filter when the second filter is mounted on the housing. Embodiments of color temperatures are described at length, below, and may be, e.g., a color temperature within a range of 2,500 to 20,000 degrees Kelvin.

An embodiment of the invention is a process of making a lamp comprising providing a plurality of filters that are mountable to the housing, with the filters providing different color temperatures for light emitted from the lamp. The color of the light after exiting the filters is preferably white. The process can also include securing a light source within a housing. The light can be white light at the source, or the filters can change the color of the light to white. The light, after being filtered by one of the filters, has a color temperature within a range of, e.g., 2,500 to 20,000 degrees Kelvin. A difference between the color temperatures may be, e.g., at least about 500, at least about 1000, at least about 1500, or at least about 5000 degrees Kelvin.

An embodiment of the invention is a light therapy method comprising administering white light from a light source to a patient and choosing one of a plurality of filters to provide a predetermined color temperature of the white light. The therapy may be a bright light therapy and/or provided for a condition chosen from the group consisting of SAD, depression, and a sleep disorder.

### BRIEF DESCRIPTION OF THE DRAWINGS

FIG. 1 is a perspective view of an embodiment of the invention;

FIG. 2 is a front elevated view of the embodiment of FIG. 1;

FIG. 3 is a cross-sectional view taken along the line 3-3′ of FIG. 2; and

FIG. 4 is an exploded view of an assembly of the embodiment of FIG. 1;

### DETAILED DESCRIPTION OF THE INVENTIONS

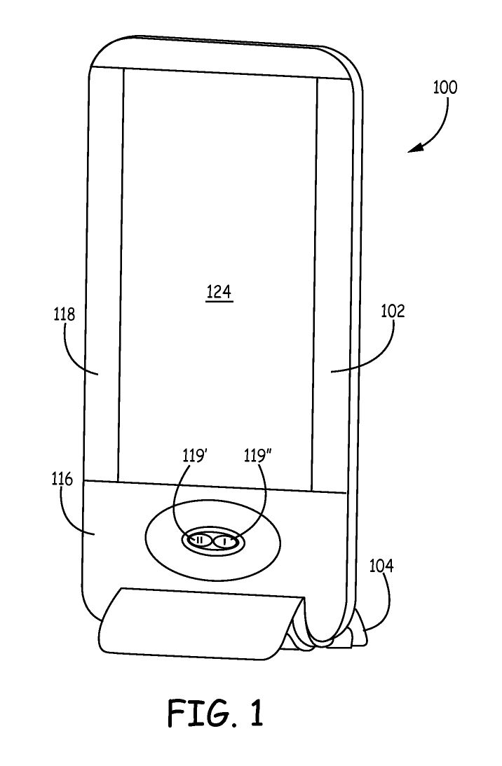

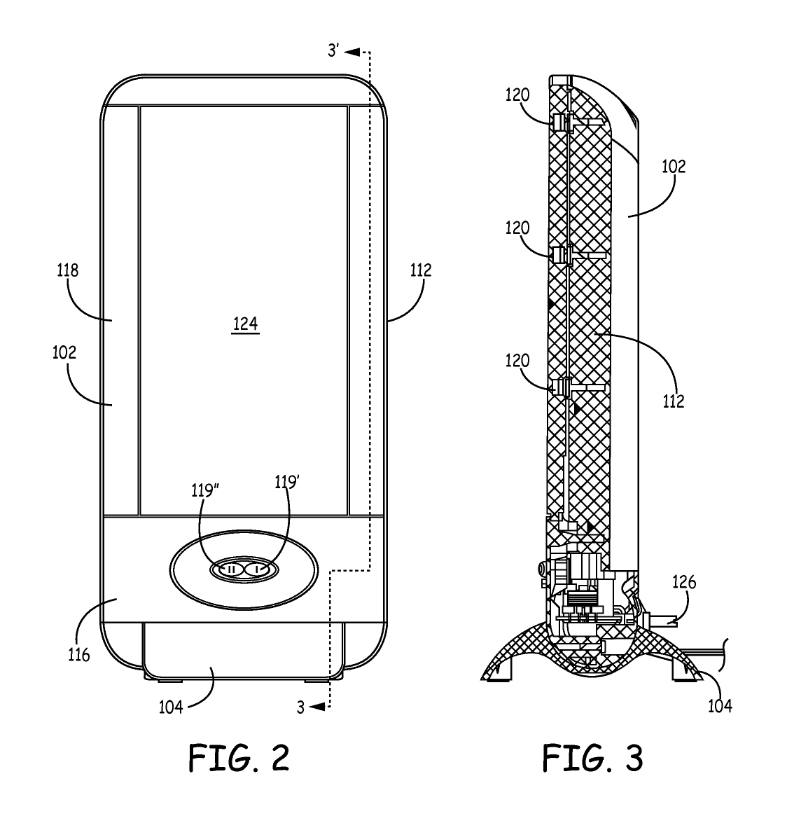

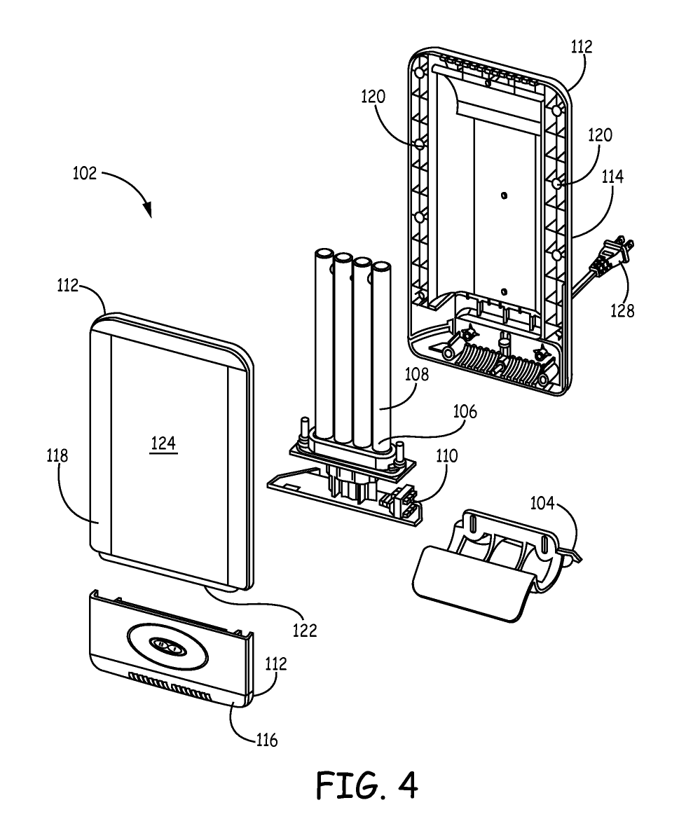

FIGS. 1-4 illustrate light therapy light 100 having lamp 102 pivotably resting in base 104. Light source 106 comprising light bulb 108 electrically connected with lighting electronics 110 is housed within housing 112. Housing 112 has back portion 114, front portion 116, and filtering portion 118. Front portion 116 has buttons 119′, 119″ for selection of different preset intensities for light source 106. Front portion 116 and back portion 114 are fastened to each other. Filtering portion 118 is reversibly fastened to back portion 114 by fasteners 120. Lip 122 guides and secures the bottom portion of filtering portion 118 into bottom housing portion 116. The term reversibly fastened means that a user may readily fasten and unfasten the items, as is known in the arts, e.g., magnetic fasteners, hook and loop materials, and threaded connections that are easily manipulated. The depicted fasteners 120 are magnetic. Filtering portion 118 comprises color temperature filter 124.

In use, a user places light 100 at a convenient location, connects plug 128 to an electrical outlet, and actuates the light by pressing one of buttons 119′, 119″. Light from the light source 106 is created at bulb 108 and passes out of housing 112 through filter 124. The filtered light is received at a user's eye and passes therein to the retina. The retina and/or other receptors inside the eye are involved in processing the filtered light in a manner that mediates the light therapy.

The light is preferably provided with a plurality of filters 124. A user may choose between the filters. The filters are interchangeably mountable on the housing, meaning that they each fit onto the housing and are designed for the user to change as desired. The filters are different, however, since they provide different properties to the filtered light. The filters preferably change a color temperature of light from the light source without substantially changing a color of the light. The color of the filtered light is preferably white, with the light being white as it is produced at the bulb or as a result of the filter removing colors/wavelengths so the filtered light is white.

The light source may be contained in a single housing that also provides a mount for the filters. The housing may have any convenient shape. It can be adapted to accommodate the various light sources that are available, and to mount filters via various schemes. There can also be a plurality of housings, e.g., for a plurality of sources and/or filters. Fasteners for securing the filters may include, e.g., magnets, screws, friction-fit, mortise-and-tenon, peg-and-hole and so forth. The filter may alternatively be mounted on a wheel, as a sheet or substantially planar object to be placed on, or secured to, a housing or housings. An embodiment is a housing with a diffuser for the light source, with a means for receiving and/or securing a filter on or over the diffuser. Various switches for controlling the light source are known, e.g., buttons, slides, toggles, dials, potentiometers.

The housing may be pivotably received in the base, as in FIGS. 1-4. A user may adjust an angle of the housing relative to the base and secure a fastener in the base to hold the housing at a fixed position. Alternatively, mounts (not shown) may be provided for mounting the housing to a surface such as a wall or ceiling.

### Color Temperature and Color

Color is a familiar concept. Various color models have been developed to describe colors, including theories that describe all colors as a specific hue (red, orange, yellow etc.) and value. The hue refers to the color spectrum. The value refers to the lightness or darkness of a color. Color temperature is a different concept that refers to light in terms of the light emitted from a black body. Color temperatures from about 2,500 to about 20,000° K. are useful in light therapies, and lights with these color temperatures are referred to as white light herein. The lights and/or filters may be further limited to a narrower range such as from about 3,000° K. to about 16,000° K., with this range being an alternative white light range. Artisans will immediately understand that all ranges and values between the explicitly stated values of 2,500 and 20,000 are contemplated. The term “color temperature” as used herein means the color temperature of a black body as well as a correlated color temperature that refers to light from non-incandescent and non-black body that is matched to the color temperature scale. The color temperature of a 60W incandescent bulb is about 2700° K., for a 100 W incandescent bulb is about 2900° K., for a halogen bulb is about 3000° K., and daylight is about 4900° K..

### Filters

Optical filters selectively transmit light of different wavelengths and are usually implemented as plane glass or plastic devices in the optical path which are either dyed/colored/pigmented in the bulk or have interference coatings. Optical filters usually belong to one of two categories: absorptive or dichroic. Absorptive optical filters selectively transmit light in a particular range of wavelengths/colors, while blocking the remainder. They can usually pass long wavelengths only, short wavelengths only, or a band of wavelengths blocking both longer and shorter wavelengths. Absorptive optical filters are usually made from either glass or plastics to which various inorganic and organic compounds have been added. These compounds absorb some wavelengths of light while transmitting others.

Suitable base/substrate materials used in these types of applications are, among others, glass, polycarbonate and acrylic [poly (methyl methacrylate)]. Suitable additives to filter various light wavelengths are ZnS, TiO₂, Ta₂O₅, HfO₂, Ln₂O₃, ZrO₂, CdS, ZnSe, Sb₂O₃, 3NaFAIF₃, CaF, SiO₂, MgF₂, NdF₃, ThF₄, HfF₄, CeO₂, ZnO, SiC, BaSO₄ among others.

The filters may be provided in sets. Sets may be chosen to include a plurality of features that each provide a white light with a different color temperature. The choice of such filters may depend on the characteristics of the light source. Some sources will provide a substantially white light so that the filters are chosen to provide a color temperature of the light without changing the light into a different color. Other sources require a color correction or removal of some wavelengths to create a white light with the filters. And, in some cases, the light source will be substantially white so that a first filter provides diffusion of the light without changing a color or a color temperature and a second filter in a set provides a change in color temperature.

Some embodiments are directed to making plastic filters that comprise different concentrations of a rare earth element so as to change a color temperature of a light without changing its color. Rare earth elements, e.g., barium, or transition elements. Absorption filters may be made from, e.g., filter glass or synthetic gels. These may be used to isolate a broad band of wavelengths and/or to block short wavelengths while transmitting longer ones. These filters are may be made in the form of glass, plastic-coated glass, acetate, or gelatin bases that have been coated, mixed, or impregnated with organic and inorganic dyes or elements obtained from both natural and synthetic sources. Among the materials used in glass and polymer filters are the rare earth transition elements, colloidal dyes (such as selenide), and other molecules having high extinction coefficients that produce reasonably sharp absorption transitions. Other processes for making light filters involve sputtering, chemical vapor deposition, and plasma-based processes. Examples of filters are, e.g., a conversion of a 3200° K. light source to 5500° K. or vice versa.

FIGS. 1-4 depict filters that are mounted in a frame. The frame, and the accompanying filter, is reversibly attachable to the housing. The term reversibly means that it is designed to be readily taken on and off by the end-user. The term frame is broad and means structure around the filter so that the filter material can be placed as intended on the device. The filters may be identical to each other in terms of size and shape, or could have differing dimensions. Alternatively, a plurality of filters may be attached to the housing and moved so that one at a time is in a filtering position on the housing to filter the light. For example, a filter wheel could be mounted (or mountable) to the housing, with the wheel being turned as desired to filter the light with a particular filter. Or a scroll or roll of filtering material could be mounted to the housing so that a user could scroll or roll the material to select a filter material. Or a plurality of plastic filter sheets could be placed in a pocket of the housing and manually selected to be placed in a filtering position. The filters may be interchangeable, meaning that they are shaped to fit the same way into the same filtering position. Further embodiments of the invention are directed to changing out the filters. Some filters change color over time as a result of being exposed to the light source. The filters tend to yellow. Embodiments include changing out the filters after a predetermined number of uses, amount of time, or time of use. Electronic counters to measure and/or track and/or record one or more of these factors on a per session or a cumulative basis may be included, e.g., on a filter, a filter frame, at a housing. Embodiments include providing one or more replacement filters in a filter set so that a replacement is at hand when needed.

### Light Sources

Fluorescent light sources are useful for providing light that is white or that can be filtered to a white color. A fluorescent lamp or fluorescent tube is a gas-discharge lamp that uses electricity to excite mercury vapor. The excited mercury atoms produce short-wave ultraviolet light that then causes a phosphor to fluoresce, producing visible light.

Alternative sources are incandescent bulbs, although the range of color temperatures available with conventional bulbs is limited. A halogen light can be useful. These are also known as a tungsten halogen lamps or quartz iodine lamps, and are incandescent lights that have a small amount of a halogen such as iodine or bromine added. The light-emitting diode (LED) or a light diode light source can also be useful. Options include combining red, green, and blue LEDs or diodes to make a white light source. Other colored light sources may alternatively be combined to make white light.

There are a variety of light sources known that can produce a white light or a light that can be filtered to a white light. Filter sets can be made to change a color temperature with these light sources. Alternatively, the light source can, in some instances, be changed to alter its color temperature. For instance, when red, green, and blue lights are combined, the mix of colors can be adjusted to change a color temperature, but only if the lights and control systems are configured with this object in mind. In many cases, conventional red, green, and blue light systems are not adaptable to change a color temperature of a white light.

### Light Therapies

Environmental light is a primary stimulus to control circadian rhythms, seasonal cycles, and neuroendocrine responses. Many studies have tested the use of light for treating Seasonal Affective Disorder (SAD), depression, sleep disorders, hormonal regulation, and eating disorders. In general, a response to light relates to its intensity, wavelength and dosage, with dosage being the product of intensity and time.

Light therapy treatment can restore circadian rhythmicity to effectively treat affective disorders and insomnia, and to increase sleep efficiency. Light therapy for SAD is generally well tolerated, with most patients experiencing clinical improvement within one to two weeks after the start of treatment. To avoid relapse, light therapy should continue through the end of the winter season until spontaneous remission of symptoms in the spring or summer. Kurlansik et al., Seasonal affective disorder, Am Fam Physician. 2012 Dec. 1; 86(11):1037-41. Light therapy is effective in treating seasonal affective disorder (SAD) and non-seasonal depression in adults, with effect sizes equivalent or superior to psychopharmacologic treatment. R. N. Golden, B. N. Gaynes, R. D. Ekstrom et al., “The efficacy of light therapy in the treatment of mood disorders: a review and meta-analysis of the evidence,” American Journal of Psychiatry, vol. 162, no. 4, pp. 656-662, 2005.

Suitable light intensities for these various conditions vary. In general, a suitable light intensity falls within a range from about 2000 lux to about 30,000 lux at the intended range of treatment, which is typically from about 6 inches to about 48 inches from the device. A lux is the SI unit of illuminance and luminous emittance, measuring luminous flux per unit area. It is equal to one lumen per square meter. Conventional wisdom teaches an intensity of about 10,000 lux for SAD but applicants have determined that that the pioneering studies that established this value did not govern and measure the actual intensity with accuracy. Measurement of light intensities requires proper equipment and controlled conditions that were not available or not known to the clinicians. Specifically, depending on the ratio of various light receptors in the eye, individuals may respond differently to different color temperatures of light therapy, impacting the total perceived brightness and biological response based on the ratio of photopic to scotopic light. Many researchers, without knowledge of the impact of scotopic light on the eye and resulting circadian system, relied solely on photopic measurements during the research such that their data did not fully capture the total amount of light as perceived by the eye and its resulting impact on the patient.

Applicants have determined that many persons do not require and/or will not comfortably tolerate an intensity of 10,000 lux. Artisans will immediately appreciate that all ranges and values between the explicitly stated limits are contemplated for the light therapy devices described herein; e.g., from about 2000 lux to about 8000 lux or 5000 lux at a range of 1 inch to 48 inches and all values in between. These intensities may be varied in combination with filtered changes in a color temperature and/or in combination with an S/P ratio; artisans will understand that all combinations and subcombinations of the same are contemplated.

### Effective Light Therapies

A lumen is the traditional measure of light intensity. The definition of the lumen is based on the response of the cone cells to light. The eye has three primary light-sensing cells in the retina, known as photoreceptors, called rods, cones and melanopsin. Cones process visual information under bright light levels. Rods mediate vision used in darkness and control the amount of light permitted to enter the eye through the pupil (scotopic conditions). Melanopsin regulates the body's circadian cycle. Melanopsin is sensitive to light intensity and is usually triggered by bright morning light.

In the past, lighting manufacturers used light meters to determine lumen output, or luminous efficacy. But these devices were based on the decades-old assumption that the cones generally mediate vision and that the light sensitive rods were relevant only in low-light, or nighttime, conditions. The role of melanopsin, discovered in 2002, was not even known. And the traditional light therapy assumptions about the light source were similarly based on the lumen.

The ratio of scotopic luminance versus photopic luminance in a light is called the S/P ratio. The S/P ratio affects the perceived brightness intensity of light. In practice, a light source can be made to appear brighter than its actual lumens. In fact, feedback inputs from rod cells control pupil size. If the rod cells can be stimulated to open the pupil, then the dose of light received in the eye can be increased without increasing the brightness (lux) of the source. Controlling the color temperature in light therapy can provide this effect. A light source with a high S/P ratio will appear brighter than the same light source with a relatively lower S/P ratio. There are other more subtle effects, such as a decrease in light scattering that reduces eye strain. All of these factors are harnessed in embodiments of the invention that manipulate light therapy light sources to provide an intended color temperature and options for users to choose a preferred color temperature. Table 1 provides correlations between color temperature and S/P ratios.

TABLE 1

| Color | |
|---|---|
| Temperature | S/P Ratio |
| 7,500 K | 2.4-2.2 |
| 6,500 K | 2.2-2.0 |
| 5,500 K | 2.0-1.8 |
| 4,500 K | 1.8-1.6 |
| 4,000 K | 1.6-1.5 |

Embodiments of the invention include a light system for treating a patient with a bright light therapy, the system comprising a lamp, a light source, and a filter set. The filter set has at least a first filter and a second filter, and are interchangeably mountable on a housing to filter light emitted from the light source. The light emitted from the lamp, before and/or after passing through one of the first or second filters, is white light. The light has a first S/P ratio after passing through the first filter when the first filter is mounted on the housing and the light has a second S/P ratio after passing through the second filter when the second filter is mounted on the housing. Embodiments include a plurality of filters with an S/P ratio from about 1.0 to about 2.5; artisans will immediately appreciate that all ranges and values between the explicitly stated limits are contemplated. For instance, a first filter provides white light at an S/P ratio of 2.2 and a second filter provides white light at an S/P ratio of 1.4. These S/P ratios may be combined with the various intensity values and ranges set forth elsewhere herein.

Embodiments of the invention include a light system for treating a patient with a bright light therapy, the system comprising a lamp, a light source, and a filter set. The filter set has at least a first filter and a second filter, and are interchangeably mountable on a housing to filter light emitted from the light source. The system, with the filters, provides a white light with temperatures from about 2,500 to about 20,000° K.; artisans will immediately understand that all ranges and values between the explicitly stated values are contemplated, e.g., a first filter in a range of from 5,000 to 10,000 degrees Kelvin after passing through the first filter and a color temperature in a second range from 3,000 to 7,000 degrees Kelvin. The filters can be made with differences in color temperatures, e.g., from about 100 to about 10,000° K.; artisans will immediately understand that all ranges and values between the explicitly stated values are contemplated, e.g., 500, 700, 1000, or 1500° K.. These color temperatures and color temperature differences may be combined with the various intensity values and ranges set forth elsewhere herein.

The light systems may be used to provide a light therapy, e.g., for one or more of the conditions described herein. A user turns on the light source and positions it within a distance that provides a therapeutic intensity. The user chooses between light color temperature-changing filters in the filter set to find a comfortable light color temperature. Moreover, the user may adjust the filters to provide a light at settings that are effective at addressing the user's specific therapeutic needs.

1. A light system comprising
a lamp that comprises a housing and a light source, and
a filter set that comprises a first filter and a second filter, with the light source being inside the housing and the filters in the filter set being mountable on the housing to filter light emitted from the light source,
wherein light emitted from the lamp, after passing through one of the first or second filters, is white light, and
wherein the light has a first color temperature after passing through the first filter when the first filter is mounted on the housing and the light has a second color temperature after passing through the second filter when the second filter is mounted on the housing.

2. The system of claim 1 wherein the light, after being filtered by the first filter or the second filter has a color temperature within a range of 2,500 to 20,000 degrees Kelvin.

3. The system of claim 1 wherein a difference between the first color temperature and the second color temperature is at least 500 degrees Kelvin.

4. The system of claim 1 wherein a difference between the first color temperature and the second color temperature is at least 5000 degrees Kelvin.

5. The system of claim 1 wherein the light has a color temperature in a first range from 5,000 to 10,000 degrees Kelvin after passing through the first filter and a color temperature in a second range from 3,000 to 7,000 degrees Kelvin after passing through the second filter, with the first filter and the second filter having a color temperature difference of more than 500 degrees Kelvin.

6. The system of claim 1 wherein the first filter and the second filter comprise an engineering plastic chosen from the group consisting of acrylates, methacrylates, polyethylene, polystyrene, polycarbonate, polyetherether ketone, acrylonitrile-butadiene-styrene, and polyurethane.

7. The system of claim 1 wherein the filters in the filter set comprise paper, plastic gels, absorbing glass filters, or a dielectric interference coating.

8. The system of claim 1 further comprising at least one more filter, wherein said filters each provide a different color temperature.

9. The system of claim 1 wherein the light source is chosen from the group consisting of a mercury bulb, a tungsten source, a LED source, a light diode source, and a fluorescent source.

10. The system of claim 1 wherein the light source comprises a plurality of LEDs that have different color outputs mixed to provide the white light.

11. The system of claim 1 wherein the light is white light before it passes through one of the filters.

12. The system of claim 1 wherein the light source emits white light.

13. The system of claim 12 wherein the filters change a color temperature of the white light without changing the white light to a non-white color.

14. The system of claim 1 wherein the light source emits light having a color that is not white.

15. The system of claim 14 wherein the color is changed to the white light after passing through one of the filters.

16. The system of claim 1 wherein the first filter and second filter are removably attachable to the housing.

17. The system of claim 1 wherein the housing is pivotably mounted on a base.

18. The system of claim 10 further comprising a fastener to prevent pivoting relative to the base.

19. The system of claim 1 wherein the lamp provides at least 1,000 lux of light at a distance of about 6 inches.

20. The system of claim 1 wherein the light source is operable at a plurality of energy settings to thereby provide a plurality of light intensities according to the energy setting chosen by a user.

21. A process of making a lamp comprising securing a light source within a housing and providing a plurality of filters mountable to the housing, with the filters providing different color temperatures for light emitted from the lamp, said light being white light.

22. The system of claim 21 wherein the light, after being filtered by one of the filters has a color temperature within a range of 2,500 to 20,000 degrees Kelvin.

23. The system of claim 21 wherein a difference between the color temperatures is at least 500 degrees Kelvin.

24. The system of claim 21 wherein the light has a color temperature in a first range from 5,000 to 10,000 degrees Kelvin after passing through a first filter and a color temperature in a second range from 3,000 to 7,000 degrees Kelvin after passing through a second filter.

25. The system of claim 21 wherein the light source is chosen from the group consisting of a mercury bulb, a tungsten source, a LED source, a light diode source, and a fluorescent source.

26. The system of claim 21 wherein the light source comprises a plurality of LEDs that have different color outputs mixed to provide the white light.

27. The system of claim 21 wherein the light source emits light having a color that is not white.

28. The system of claim 21 wherein the lamp provides at least 1,000 lux of the light at a distance of about 6 inches.

29. A light therapy method comprising administering white light from a light source to a patient and choosing one of a plurality of filters to provide a predetermined color temperature of the white light.

30. The therapy of claim 29 being provided for a condition chosen from the group consisting of seasonal affective disorder, depression, and a sleep disorder.

31. The therapy of claim 29 wherein, when choosing among the plurality of filters, a difference between a color temperature of a first filter and a second filter is at least 500 degrees Kelvin.

32. The therapy of claim 29 wherein the patient receiving the therapy chooses among the filters and installs the chosen filter on a lamp that comprises the light source.

33. The therapy of claim 29 wherein the lamp provides at least 1,000 lux of the light at a distance of about 6 inches.
